(19) United States
(12) Patent Application Publication
(10) Pub. No.: US 2006/0135948 A1
(43) Pub. Date: Jun. 22, 2006

(54) Multi-exchange catheter guide member with improved seal

(75) Inventor: Ashish Varma, Galway (IE)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., a Delaware Corporation, Santa Rosa, CA (US)

(21) Appl. No.: 11/015,272

(22) Filed: Dec. 16, 2004

(51) Int. Cl. A61M 25/00

(52) U.S. Cl. 604/523; 604/264

(57) ABSTRACT

A catheter and a guidewire exchange system includes a catheter and a guide member. The catheter includes a lumen extending through the shaft and sized to receive the guidewire, and a longitudinal guideway enabling transverse access from the shaft exterior surface to the lumen. The guide member includes a housing, a catheter passageway extending through the housing and adapted to slidably receive the catheter, a guidewire passageway extending from one end of the housing into the catheter passageway and including a tube adapted to merge the guidewire transversely through the guideway and into the first lumen, and a user-activated device positioned in the guidewire passageway and including a clamping body adapted to clamp the guidewire and thereby secure the guidewire in the guidewire passageway.

### TECHNICAL FIELD

The present invention generally relates to medical catheters and medical apparatuses involving medical catheters. The present invention more particularly relates to seals for guide members of Multi-Exchange catheters.

### BACKGROUND

Cardiovascular disease, including atherosclerosis, is a leading cause of death in the U.S. The medical community has developed a number of methods and devices for treating coronary heart disease, some of which are specifically designed to treat the complications resulting from atherosclerosis and other forms of coronary arterial narrowing.

One method for treating atherosclerosis and other forms of coronary narrowing is percutaneous transluminal coronary angioplasty, commonly referred to as “angioplasty” or “PTCA.” The objective in angioplasty is to enlarge the lumen of the affected coronary artery by radial hydraulic expansion. The procedure is accomplished by inflating a balloon of a balloon catheter within the narrowed lumen of coronary artery.

In addition to PTCA, catheters are used for delivery of stents or grafts, therapeutic drugs (such as anti-vaso-occlusion agents or tumor treatment drugs) and radiopaque agents for radiographic viewing. Other uses for such catheters are well known in the art.

The anatomy of coronary arteries varies widely from patient to patient. Often a patient's coronary arteries are irregularly shaped, highly tortuous and very narrow. The tortuous configuration of the arteries may present difficulties to the physician in proper placement of a guidewire, and advancement of a catheter to a treatment site. A highly tortuous coronary anatomy typically will present considerable resistance to advancement of the catheter over the guidewire.

Therefore, it is important for a catheter to be highly flexible. However, it is also important for a catheter shaft to be stiff enough to push the catheter into the vessel in a controlled manner from a position far away from the distalmost point of the catheter.

Catheters for PTCA and other procedures may include a proximal shaft, a transition section and a distal shaft having a flexible distal tip. In particular, the catheters have a proximal shaft, which is generally rigid for increased pushability and a more flexible distal shaft with a flexible distal tip for curving around particularly tortuous vessels. The proximal shaft may be made stiff by the insertion of a thin biocompatible tube, such as a stainless steel hypotube, into a lumen formed within the proximal shaft. The transition section is the portion of the catheter between the stiffer proximal shaft and the more flexible distal shaft, which provides a transition in flexibility between the two portions.

With some types of catheter construction, when an increase in resistance occurs during a procedure there is a tendency for portions of the catheter to collapse, buckle axially or kink, particularly in an area where flexibility of the catheter shaft shifts dramatically. Consequently, the transition section is often an area where the flexibility of the catheter gradually transitions between the stiff proximal shaft and the flexible distal shaft. It is known in the art to create a more gradual flexibility transition by spiral cutting a distal end of the hypotubing used to create stiffness in the proximal shaft. Typically, the spiral cut is longitudinally spaced father apart at the hypotube proximal end creating an area of flexibility, and longitudinally spaced closer together at the hypotube distal end creating an area of even greater flexibility.

In a typical PTCA procedure, it may be necessary to perform multiple dilatations, for example, using various sized balloons. In order to accomplish the multiple dilatations, the original catheter must be removed and a second catheter tracked to the treatment site. When catheter exchange is desired, it is advantageous to leave the guidewire in place while the first catheter is removed to properly track the second catheter.

Two types of catheters commonly used in angioplasty procedures are referred to as over-the-wire (OTW) catheters and rapid exchange (RX) catheters. A third type of catheter with preferred features of both OTW and RX catheters, which is sold under the trademarks MULTI-EXCHANGE, ZIPPER MX, ZIPPER, MX and/or MXII, is discussed below. An OTW catheter's guidewire lumen runs the entire length of the catheter and may be positioned next to, or enveloped within, an inflation shaft. Thus, the entire length of an OTW catheter is tracked over a guidewire during a PTCA procedure. A RX catheter, on the other hand, has a guidewire lumen that extends within only the distalmost portion of the catheter. Thus, during a PTCA procedure only the distalmost portion of a RX catheter is tracked over a guidewire.

If a catheter exchange is required while using a standard OTW catheter, the user must add an extension wire onto the proximal end of the guidewire to maintain control of the guidewire, slide the catheter off of the extended guidewire, slide the new catheter onto the guidewire and track back into position. Multiple operators are required to hold the extended guidewire in place while the original catheter is exchanged in order to maintain its sterility.

A RX catheter avoids the need for multiple operators when exchanging the catheter. With a rapid exchange catheter, the guidewire runs along the exterior of the catheter for all but the distalmost portion of the catheter. As such, the guidewire can be held in place without an extension when the catheter is removed from the body. However, one problem associated with RX catheters is the guidewire, and most of the catheter, must be removed from the body in order to exchange guidewires. Essentially the procedure must then start anew because both the guidewire and the catheter must be retracked to the treatment site. An OTW catheter, with the guidewire lumen extending the entire length of the catheter, allows for simple guidewire exchange.

A balloon catheter capable of both fast and simple guidewire and catheter exchange is particularly advantageous. A catheter designed to address this need is sold by Medtronic Vascular, Inc. of Santa Rosa, Calif. under the trademarks MULTI-EXCHANGE, ZIPPER MX, ZIPPER, MX and/or MXII (hereinafter referred to as the “MX catheter”). An MX catheter is disclosed in U.S. Pat. No. 4,988,356 to Crittenden et al.; co-pending U.S. patent application Ser. No. 10/116,234, filed Apr. 4, 2002; co-pending U.S. patent application Ser. No. 10/251,578, filed Sep. 18, 2002; co-pending U.S. patent application Ser. No. 10/251,477, filed Sep. 20, 2002; co-pending U.S. patent application Ser. No. 10/722,191, filed Nov. 24, 2003; and co-pending U.S. patent application Ser. No. 10/720,535, filed Nov. 24, 2003, all of which are incorporated by reference in their entirety herein.

The MX catheter includes a catheter shaft having a guidewire lumen positioned side-by-side with an inflation lumen. The MX catheter also includes a longitudinal cut that extends along the catheter shaft and that extends radially from the guidewire lumen to an exterior surface of a catheter shaft. A guide member through which the shaft is slidably coupled cooperates with the longitudinal cut such that a guidewire may extend transversely into or out of the guidewire lumen at any location along the longitudinal cut's length. By moving the shaft with respect to the guide member, the effective over-the-wire length of the MX catheter is adjustable.

The guidewire is threaded into a guidewire lumen opening at the distal end of the catheter and out through the guide member. The guidewire lumen envelopes the guidewire as the catheter is advanced into the patient's vasculature while the guide member and guidewire are held stationary. Furthermore, the indwelling catheter may be removed by withdrawing the catheter from the patient while holding the proximal end of the guidewire and the guide member in a fixed position. When the catheter has been withdrawn to the point where the distal end of the cut has reached the guide member, the distal portion of the catheter over the guidewire is of a sufficiently short length that the catheter may be drawn over the proximal end of the guidewire without releasing control of the guidewire or disturbing its position within the patient.

During some catheter advancing and retracting processes, including a catheter exchange, it can be difficult to hold the guidewire proximal end and the guide tool in a fixed position with one hand, while retracting or advancing the catheter with the other hand. Once the guidewire is positioned in a desired region of the patient's body, it is important to maintain that guidewire position to enable the present catheter or a replacement catheter to quickly advance through an occluded or tortuous vein.

While the MX catheter provides many advantages over RX catheters, like an RX catheter, the proximal shaft may not be completely secured in the hemostasis valve. For example, in a typical dye injection the physician may pull a slight negative pressure to ensure that no air bubbles are in the system. However, if the physician pulls a very heavy vacuum, there remains the possibility that air may enter the patient through the hemostasis valve if it is not sealed sufficiently. Similarly, RX catheters used with passive/active gaskets in a hemostasis valve may also be susceptible to air entering if the gasket is not closed properly and a very heavy vacuum is drawn.

Accordingly, it is desirable to provide an apparatus that can reduce or eliminate the opportunity for unwanted air aspiration. In addition, it is desirable to provide such an apparatus that does not slow down guidewire insertion or other medical processes involving the catheter. Furthermore, other desirable features and characteristics of the present invention will become apparent from the subsequent detailed description and the appended claims, taken in conjunction with the accompanying drawings and the foregoing technical field and background.

### BRIEF SUMMARY

A system is provided to exchange a catheter and/or a guidewire. The system includes a catheter and a guide member. The catheter includes a lumen extending through the shaft and sized to receive the guidewire, and a longitudinal guideway enabling transverse access from the shaft exterior surface to the lumen. The guide member includes a housing, a catheter passageway extending through the housing and adapted to slidably receive the catheter, a guidewire passageway extending from one end of the housing into the catheter passageway and including a tube adapted to merge the guidewire transversely through the guideway and into the first lumen, and a user-activated device positioned in the guidewire passageway and including a clamping body adapted to clamp the guidewire and thereby secure the guidewire in the guidewire passageway.

### BRIEF DESCRIPTION OF THE DRAWINGS

The present invention will hereinafter be described in conjunction with the following drawing figures, wherein like numerals denote like elements, and

FIG. 1 is a perspective view of a guide member with a guide wire extending through a guide member and into a catheter according to the present invention;

FIGS. 2A-D are cross sectional views of a catheter at points A-A, B-B, C-C, and D-D illustrated in FIG. 1;

FIG. 3 is a perspective cross sectional view of an oval proximal shaft;

FIG. 4 is a cross sectional view of a circular proximal shaft;

FIG. 5 is a sectional view of a guide member and its components according to the present invention;

FIG. 6 is a sectional view of the proximal guidewire pathway including the guidewire port and the tube, along with a tapered seal secured in the guidewire port according to an embodiment of the present invention;

FIG. 7 is a sectional view of the proximal guidewire pathway including the guidewire port and the tube, along with a compression seal secured in the guidewire port according to an embodiment of the invention;

FIG. 8 is a perspective view of a flap seal according to an embodiment of the invention; and

FIG. 9 is a sectional view of the proximal guidewire pathway including the guidewire port and the tube, along with the flap seal secured in the guidewire port and a switch extending through an opening in the guide member according to an embodiment of the present invention.

### DETAILED DESCRIPTION

The following detailed description is merely exemplary in nature and is not intended to limit the invention or the application and uses of the invention. Furthermore, there is no intention to be bound by any expressed or implied theory presented in the preceding technical field, background, brief summary or the following detailed description.

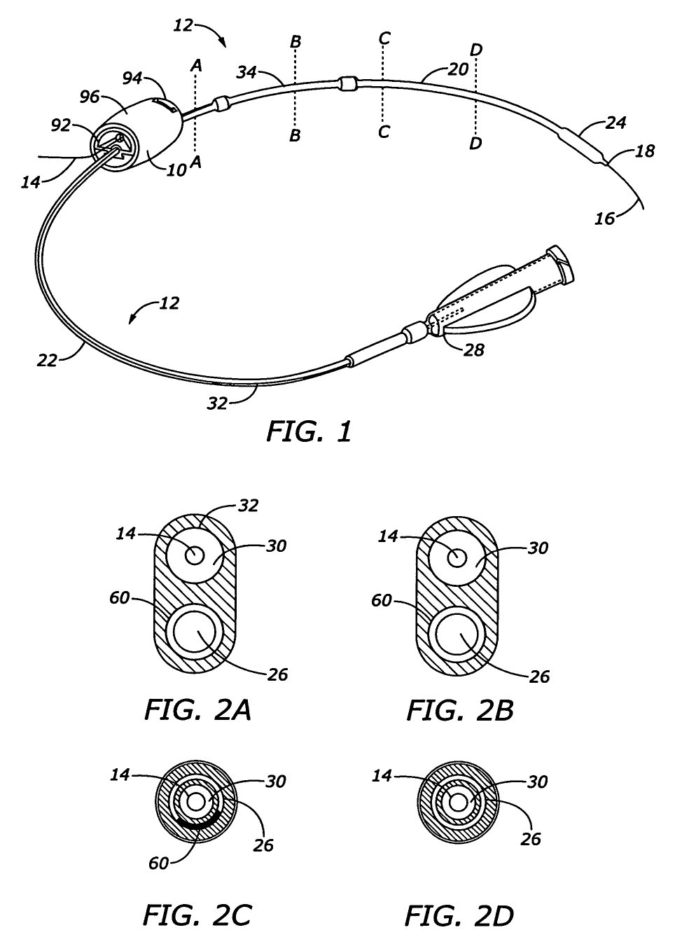

The present invention is used with an MX catheter, an exemplary embodiment of which is illustrated in FIG. 1. The catheter 12 includes an elongate, flexible, cylindrical main body having a distal shaft 20 and a proximal shaft 22. According to the present embodiment, the catheter 12 is a delivery catheter for such procedures as PTCA or stent delivery and has a balloon 24 mounted around the catheter body near the catheter distal end 18. The balloon 24 may be inflated and deflated through the catheter inflation lumen 26. The inflation lumen 26 communicates with a fitting 28 at the catheter proximal end, and extends the catheter length to terminate in communication with the balloon interior at the catheter distal end 18. The catheter 12 also includes a guidewire lumen 30 that receives the guidewire 14 and extends the entire catheter length. A longitudinal cut extends into the guidewire lumen 30 along the length of most of the proximal shaft 22 to form a guideway 32. The proximal shaft distal section 34 does not include the guideway 32. The guidewire lumen 30 and the inflation lumen 26 are coaxially arranged in the distal shaft 20 according to the present embodiment.

The present invention includes a guide member for the MX catheter 12. FIG. 1 depicts a guide member 10 according to an embodiment of the invention, with a guide wire 14 extending through the guide member 10 and into the MX catheter 12. FIGS. 2A to 2D are cross sections of the catheter 12 at points A-A, B-B, C-C, and D-D along the catheter length. The guide member 10 serves as a juncture in which the catheter 12 and guidewire 14 may be merged or separated so that the guidewire portion that extends proximal to the guide member 10 is separated from the catheter 12, and the guidewire portion that is located distal to the guide member 10 is contained and housed within the catheter, although the guidewire distal end 16 may protrude out of the catheter distal end 18.

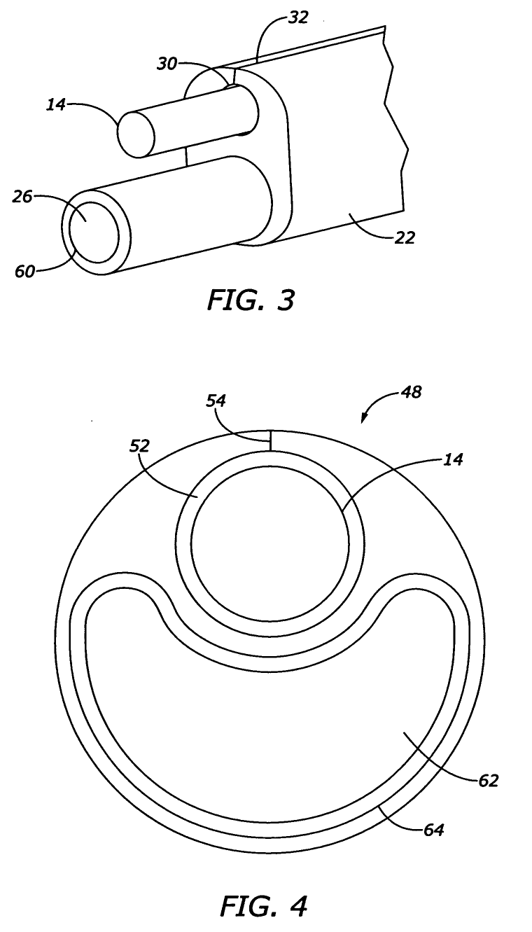

The catheter proximal shaft 22 described above can be modified to suit various needs. For example, the proximal shaft can be a tri-lumen shaft to provide passage for various drugs, fluids, wires, or other necessary compositions or equipment. Further, the proximal shaft may be oval, circular, or other suitable shape. FIG. 3 is a perspective cross sectional view of an oval proximal shaft 22 according to one embodiment of the invention, and FIG. 4 is a cross sectional view of a circular proximal shaft 48 according to another embodiment of the invention. Each of the proximal shafts 22, 48 has a respective guidewire lumen 30, 52 that is accessible through a guideway 32, 54 located along the proximal shaft length. Each of the proximal shafts 22, 48 also includes an inflation lumen 26, 62 that extends side by side with the guidewire lumen 30, 52 along the proximal shaft length. The inflation lumens 26, 62 are preferably supported by a stiffening member 60, 64 such as a hypotube. The inflation lumen 62 in the embodiment depicted in FIG. 4 is crescent shaped and the hypotube stiffening member 64 also is formed in the same shape to withstand force transmission along the catheter length. The stiffening members may further include a transition section at their respective distal sections in conjunction with a transition between the relatively stiff proximal shaft to the relatively flexible distal shaft and avoid shaft kinking at the junction therebetween. For example, the hypotube 60 may be skived at its distal end, with the skived portion extending into the distal section as depicted in FIG. 2C.

Returning to FIG. 1, the proximal shaft 22 can be formed from suitable biomedical grade materials such as polyethylene, cross-linked polyethylene, polyolefins, polyamides, blends of polyamides and polyolefins, fluoropolymers, polyesters, polyketones, polyimides, polysulphones, polyoxymethylenes, and compatibilizers based on polyolefins, including grafted polyolefins, and other comparable materials. A lubrication additive may also be used with any polymer and may include polyethylene micro-powders, fluoropolymers, silicone based oils, fluoro-ether oils, molybdenum disulphide and polyethylene oxide. Additionally, a reinforcing additive may be used such as nano-clays, graphite, carbon fibers, glass fibers, and polymeric fibers. The distal shaft 20 can be made of a suitable polyethylene or polyolefin that readily bonds to the proximal shaft 22.

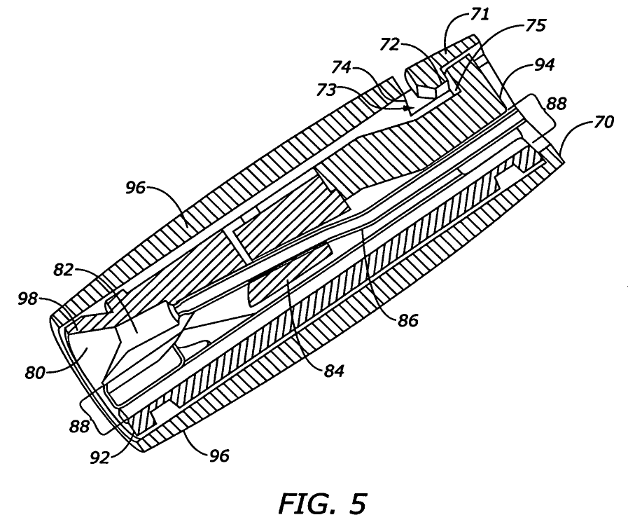

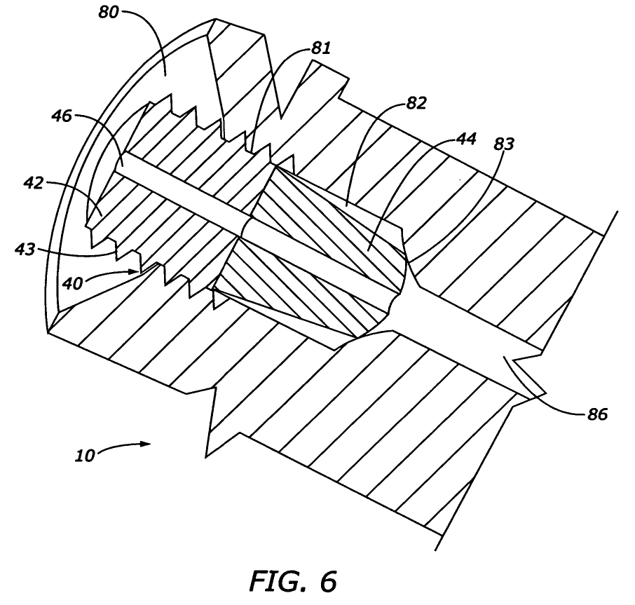

Turning now to FIG. 5, the guide member 10 and its components will be discussed according to one embodiment of the invention. The guide member 10 surrounds the proximal shaft 22 and includes proximal and distal ends 92, 94. An outer tubular member 96 freely rotates around an inner main body 98 and hence is decoupled from the inner main body 98. An inwardly extending distal annular wall 70 prevents the main body 98 from slipping out of the outer member 96. A retaining clip 71 includes a tab 72 that extends into a space 73 formed by two main body walls 74, 75. Additional tabs may be used as necessary to retain the inner main body 98 within the tubular member 96.

The guide member main body 98 includes a catheter passageway 88 extending longitudinally in a generally straight line from the guide member proximal end 92 to the guide member distal end 94. A guidewire passageway 80 extends distally from the guide member proximal end 92 through an entrance port 82 into a tube 86 and then into the catheter guidewire lumen 30, although the catheter is not depicted in FIG. 5. The passageway 80 is configured to slidingly receive the proximal shaft 22, and has a cross sectional shape that approximates the proximal shaft shape, whether the proximal shaft is circular, oval, triangular, shamrock shaped, or otherwise shaped. The passageway 80 enlarges in a central area to provide space for a keel 84 that is aligned with the passageway 80 and positioned to spread the catheter guideway 32 and extend into the catheter guidewire lumen 30 to enable guidewire insertion during use.

The entrance port 82 is configured to mate with a conventional wire introducer tool and is tapered to aid in loading such a tool. The tube 86 may vary in its length, although in an exemplary embodiment of the invention the tube 86 extends through the catheter guidewire lumen 30 approximately thirty-five millimeters past the guide member distal end 94. The tube 86 may be formed from a flexible material such as a polyimide, and particularly the tube region that extends through the catheter guidewire lumen 30. In one embodiment of the invention the tube region that introduces the guidewire 14 into the guidewire lumen 30 may be substantially rigid to provide the necessary support for the guidewire 14.

The guide member 10 is made of blends of polyamides and polyolefins in an exemplary embodiment of the invention. Other exemplary materials include ceramics, metals such as stainless steel, and other polymers such as polyamides and liquid crystal polymers. Lubrication additives such as polyethylene micro-powders, fluoropolymers, silicone-based oils, fluoro-ether oils, molybdenum disulphide, and polyethylene oxide may be included. Reinforcing additives such as nano-clays, graphite, carbon fibers, glass fibers, polyesters, polyketones, polyimides, polysulphones, polyoxymethylenes, polyolefins, cross-linked polyolefins may also be included, along with compatibilizers based on polyolefins, such as grafted polyolefins, ceramics, and metals.

An exemplary guide member operation will now be described, although the procedures in the following description clearly set forth only one of many operations enabled by the guide member 10. After the guidewire 14 and a guide catheter (not shown) are inserted into a patient, the catheter 12 is inserted with a backloading operation. The guidewire 14 is inserted into the catheter distal end 18 and threaded proximally through the guidewire lumen 30 until the guidewire tube 86 captures the guidewire proximal end and directs it into the passageway 80 and then out of the guide member proximal end 92. This procedure can be accomplished with the guide member 10 adjacent the catheter guideway distal end. As the distal shaft 20 enters the patient, the guide member 10 will reach the hemostatic valve (not shown). The guide member 10 is not intended to enter the valve and is seated adjacent to the valve. The proximal shaft 22 is then advanced through the guide member, and the keel 84 engages the catheter guideway 32. After the catheter 12 is inserted, the hemostatic valve may be closed down on the catheter shaft at a region that is distal to the guide member 10. Since the tube 86 extends in to the distal shaft 20, it is subjected to the valve clamping force. If a wire change is required, one simply withdraws the guidewire 14 from the guide member 10 as the guide member 10 is seated against the valve and as the proximal shaft 22 remains in the patient. A new guidewire is then inserted into the catheter through the passageway 80. If a catheter exchange is required, one simply holds the guidewire 14 in place and begins moving the proximal shaft 22 proximally through the guide member. Another catheter may then be backloaded onto the guidewire 14 and introduced into the patient as described above.

In order to overcome the potential for air aspiration through the guidewire lumen 30 at the catheter proximal end, the passageway 80 is adapted to include a seal that prevents or minimizes air movement through the passageway. FIG. 6 is a sectional view of the proximal guidewire pathway 80 including the guidewire port 82 and the tube 86, along with a tapered seal 40 secured in the guidewire port 82. The seal 40 has an opening 46 extending therethrough that is sized to be slightly wider than the guidewire diameter in order to enable substantially frictionless guidewire advancement and retraction. The opening 46 is also narrow enough to substantially eliminate airflow through the opening 46 during guidewire advancement.

The seal 40 includes a rigid cylindrical body 42 that secures the seal 40 in the guidewire entrance port 82. The cylindrical body 42 includes a threaded outer surface 43 that rotatably engages with threads 81 in the guidewire entrance port 82. The seal 40 also includes a tapered tip 44 that is formed from an elastomer material. The tip 44 has an outer surface in the form of a truncated cone. When the seal 40 is rotated in a tightening direction, the seal can be secured in the guidewire entrance port 82 until the tip 44 abuts a tapered tube entrance 83 as illustrated in FIG. 6. When the tip 44 is merely abutting the tube entrance 83, the opening 46 is wide enough to enable substantially frictionless guidewire advancement and retraction and to substantially eliminate airflow through the opening 46.

If a user wishes to completely eliminate airflow through the opening 46 or to clamp the guidewire in a desired position, the seal 40 can be further rotated in a tightening direction. Further tightening causes the elastomer material in the tip 44 to change shape and constrict the opening 46 around the guidewire 14. The seal 40 can be rotated until the guidewire 14 is tightly secured in its position, and a substantially airtight seal is provided around the guidewire 14. Likewise, if a user wishes to unclamp the guidewire, the seal 40 can be rotated in a loosening direction until the elastomer material in the tip 44 retains its original shape and the opening 46 retains its original diameter.

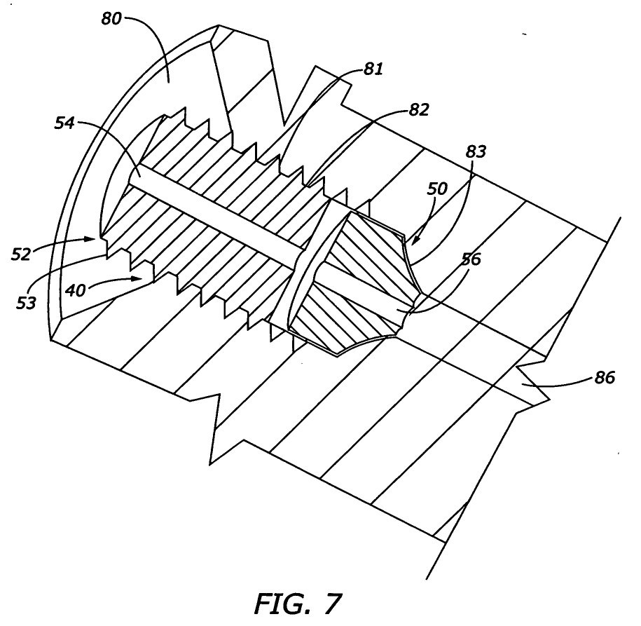

FIG. 7 is a sectional view of the proximal guidewire pathway 80 including the guidewire port 82 and the tube 86, along with a compression seal 50 secured in the guidewire port 82 according to another embodiment of the invention. The compression seal 50 has an opening 56 extending therethrough that is sized to be slightly wider than the guidewire diameter in order to enable substantially frictionless guidewire advancement and retraction. The opening 56 is also narrow enough to substantially eliminate airflow through the opening 56 during guidewire advancement.

The seal 50 is depicted in FIG. 7 to have a proximal cylindrical portion and a distal tapered region to illustrate that the seal 50 can be formed to closely match the entrance port contours. However, the compression seal 50 can be formed to have an entirely cylindrical shape if the seal rests against a lateral wall. Further, the compression seal can be formed to rest against any entrance port surface or other surface that provides a counter force that directly opposes a seal tightening force. The main difference between the compression seal 50 and the tapered seal 40 is the compression seal is shaped to have the seal 50 primarily compressed in a longitudinal direction when subjected to a tightening force, and expanded in a lateral direction as an effect of the longitudinal compression. The lateral compression causes the opening 56 to constrict and form a substantially airtight seal around the guidewire and also clamp the guidewire in place. In contrast, the tapered seal 40 is shaped to be compressed primarily in a lateral direction.

As with the tapered seal 40, the compression seal 50 is formed from an elastomer material that changes shape when compressed to constrict the guidewire opening 56, and retains its original shape when the compression force is removed. A tightening tool 52 includes a threaded rigid cylindrical body that secures the seal 50 in the guidewire entrance port 82. The cylindrical body 52 includes a threaded outer surface 53 that rotatably engages with threads 81 in the guidewire entrance port 82. When the tool 52 is rotated in a tightening direction, the tool 52 compresses the seal 50 in a primarily longitudinal direction. The longitudinal compression causes the seal to expand in a lateral direction. The lateral expansion causes the guidewire opening 56 to constrict, forming a substantially airtight seal with the guidewire 14 and securing the guidewire 14 in place. When the tool 52 is rotated in a loosening direction, the seal 50 retains its original shape.

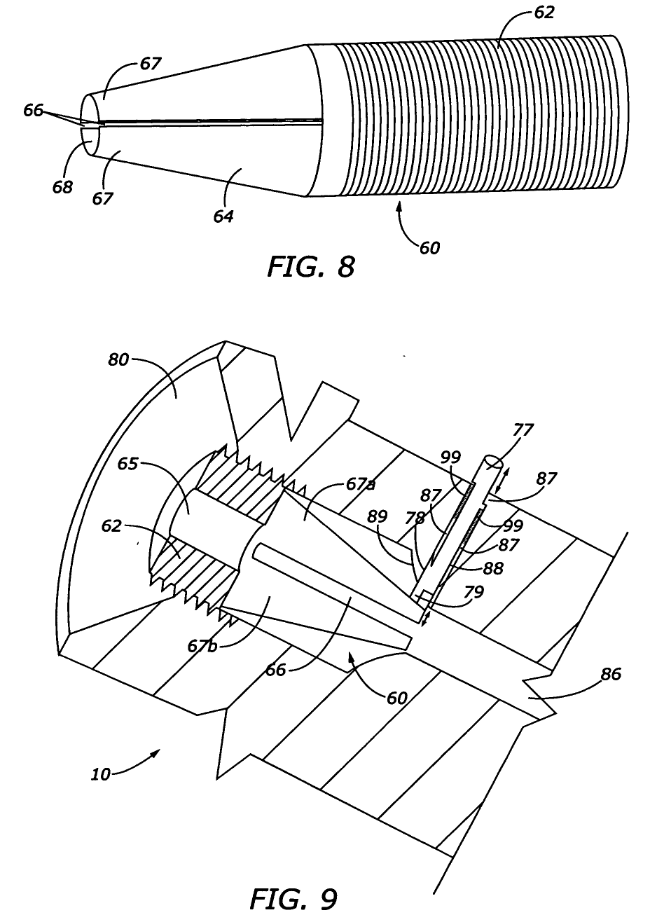

Referring now to FIG. 8, a perspective view of a flap seal 60 according to another embodiment of the invention reveals a two part structure. The first part is a threaded rigid cylindrical body 62 similar to the threaded structures 42, 52 described above. The threaded body 62 engages with threads 81 in the guidewire entrance port 82 to secure the seal 60 in place. The threaded body 62 includes an opening (not shown) through which the guidewire 14 extends. The second part of the seal 60 is a tapered body 64 that is essentially formed in the shape of a cone with a truncated tip that defines an opening 68 that is continuous with the opening (not shown) in the threaded body 62. A plurality of longitudinal slits 66 are formed in the tapered body 64. The slits 66 separate the distal tapered body 64 into a plurality of flaps 67. The flaps are formed from an elastomer or other flexible material, and are biased in a position such that they do not touch one another. Although only two flaps 67 are depicted in FIG. 8, additional slits may be included to separate the tapered body 64 into additional flaps.

During guidewire advancement and retraction, the flaps 67 are spread apart enough to allow a substantially frictionless guidewire pathway. The flaps 67 are biased in a separated position, but still substantially limit or prevent airflow into the guidewire passageway 80 during guidewire advancement. The guidewire can be secured in place by rotating the threaded body 62 in a tightening direction, causing the flaps 67 to contact the tube entrance 83 and be pressed around the guidewire 14. In the tightened position, the flaps 67 secure the guidewire in place and also further provide an airflow seal. The flaps 67 return to their biased separated position when the threaded body 62 is rotated in a loosening direction.

The above descriptions of various seals include the use of a rotatable cylindrical body that is threadedly engaged with the guidewire entrance port 82 to provide a user with ease and efficiency in the process of clamping or freeing the guidewire and limiting airflow through the opening 46. However, it is within the purview of the invention that the seal 40 and any of the other seals described herein can be secured and manipulated by a user using any suitable conventional clamp or securing device or material. FIG. 9 is a sectional view of the proximal guidewire pathway 80 including the guidewire port 82 and the tube 86, along with the flap seal 60 secured in the guidewire port 82, although any of the seals discussed above may be used in accordance with the embodiment illustrated in FIG. 9. Instead of rotating the seal 60 to clamp the guidewire and form a seal, an actuating switch or button 77 is coupled to the guide member 10 and is secured in a through hole 88 using tabs 87 and springs 99 to perform the clamping function.

The seal 60 is secured in the guidewire port 82 using the threaded body 62 or any suitable securing mechanism. With the seal 60 in place, the actuating button 77 is configured to be pressed to a locking position such that a first end 79 of the button presses one of the flaps 67a toward an opposing flap 67b to clamp the guidewire 14 and provide a substantially airtight seal with the guidewire 14. The button 77 can be equipped with a hook 78 or other structure to latch the button 77 in the locking position in an exemplary embodiment of the invention. The hook 78 can engage with a tab 89 or any other structure that is integral with or otherwise combined with the guide member 10. To release the button 77 from the locking position, the user need only press the button 77 again and allow the hook to disengage with the tab.

In an embodiment similar to that depicted in FIG. 9, the flap 67a is formed using an elastomer or another flexible material, and the flap 67b is a rigid material. Alternatively, the flap 67b can extend into the tube 86, and the button 77 can be positioned above the tube 86 and the flap 67b so that the tube 86 and the flap 67b together clamp the guidewire 14 and provide a substantially airtight seal with the guidewire 14 when the button 77 is in the locking position. Further, the advantages provided by the embodiment illustrated in FIG. 9 can be accomplished using any of the other seals described herein and equivalent seals together with the actuating button 77.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing the exemplary embodiment or exemplary embodiments. It should be understood that various changes can be made in the function and arrangement of elements without departing from the scope of the invention as set forth in the appended claims and the legal equivalents thereof.

What is claimed is:

1. A catheter and guidewire exchange system, comprising:
a catheter, comprising:
an elongate shaft having an exterior surface, a proximal end, and a distal end,
a first lumen extending through the shaft from the shaft proximal end to the shaft distal end, and sized to receive a guidewire, and
a longitudinal guideway extending distally from the shaft proximal end, and enabling transverse access from the shaft exterior surface to the first lumen; and
a guide member, comprising:
a housing having a proximal end and a distal end,
a catheter passageway extending through the housing from the proximal end to the distal end and adapted to slidably receive the catheter,
a guidewire passageway extending from the housing proximal end into the catheter passageway, and comprising a tube adapted to merge the guidewire transversely through the guideway and into the first lumen, and
a user-activated device positioned in the guidewire passageway and comprising a clamping body adapted to clamp the guidewire and thereby secure the guidewire in the guidewire passageway.

2. The system according to claim 1, wherein the clamping device is further adapted to reduce airflow through the guidewire passageway.

3. The system according to claim 1, wherein the guidewire passageway further comprises a guidewire entrance port that is positioned proximal to the tube, and the clamping device is secured within the guidewire entrance port.

4. The system according to claim 1, wherein the guidewire passageway further comprises a threaded region, and the clamping device further comprises a rotatable cylindrical body having a threaded outer surface that is engaged with the threaded region, wherein the clamping body clamps the guidewire when the cylindrical body is rotated in a first direction.

5. The system according to claim 1, wherein the clamping body has an annular cross section and an aperture adapted to slidably receive the guidewire.

6. The system according to claim 5, wherein the clamping body further comprises tapered tip.

7. The system according to claim 6, wherein the guidewire passageway further comprises an aperture that is adapted to receive and primarily laterally compress the tapered tip and thereby constrict the aperture around the guidewire.

8. The system according to claim 5, wherein the guidewire passageway comprises a compression wall that is adapted to primarily longitudinally compress the clamping body and thereby constrict the aperture around the guidewire.

9. The system according to claim 1, wherein the clamping body comprises a cone-shaped segment having a central aperture adapted to slidably receive the guidewire, and a plurality of longitudinal slits dividing the cone-shaped segment into a plurality of flaps.

10. The system according to claim 9, wherein the plurality of flaps are biased apart from one another, and the guidewire passageway further comprises an aperture that is adapted to receive the cone-shaped segment and bring the flaps in contact with one another and thereby constrict the aperture around the guidewire.

11. The system according to claim 1, wherein the guide member further comprises:
an aperture providing transverse access to the guidewire passageway from outside the guide member;
a mechanical switch extending through the aperture and comprising a first end adapted to press against the clamping body with a force that is sufficient to clamp the guidewire.

12. The system according to claim 11, wherein the mechanical switch is biased away from the clamping body and requires an unbiasing force to press against the clamping body.

13. The system according to claim 13, wherein the mechanical switch is biased using at least one spring.

14. An apparatus for advancing and retracting a guidewire and a catheter having a lumen, an exterior surface, and a longitudinal guideway that enables transverse access from the catheter exterior surface to the lumen in a patient, the apparatus comprising:
a housing having a proximal end and a distal end,
a catheter passageway extending through the housing from the proximal end to the distal end and adapted to slidably receive the catheter,
a guidewire passageway extending from the housing proximal end into the catheter passageway, and comprising a tube adapted to merge the guidewire transversely through the guideway and into the first lumen, and
a user-activated device positioned in the guidewire passageway and comprising a clamping body adapted to clamp the guidewire and thereby secure the guidewire in the guidewire passageway.

15. The apparatus according to claim 14, wherein the clamping device is further adapted to reduce airflow through the guidewire passageway.

16. The apparatus according to claim 14, wherein the guidewire passageway further comprises a guidewire entrance port that is positioned proximal to the tube, and the clamping device is secured within the guidewire entrance port.

17. The apparatus according to claim 14, wherein the guidewire passageway further comprises a threaded region, and the clamping device further comprises a rotatable cylindrical body having a threaded outer surface that is engaged with the threaded region, wherein the clamping body clamps the guidewire when the cylindrical body is rotated in a first direction.

18. The apparatus according to claim 14, wherein the clamping body has an annular cross section and an aperture adapted to slidably receive the guidewire.

19. The apparatus according to claim 18, wherein the clamping body further comprises tapered tip.

20. The apparatus according to claim 19, wherein the guidewire passageway further comprises an aperture that is adapted to receive and primarily laterally compress the tapered tip and thereby constrict the aperture around the guidewire.

21. The apparatus according to claim 18, wherein the guidewire passageway comprises a compression wall that is adapted to primarily longitudinally compress the clamping body and thereby constrict the aperture around the guidewire.

22. The apparatus according to claim 14, wherein the clamping body comprises a cone-shaped segment having a central aperture adapted to slidably receive the guidewire, and a plurality of longitudinal slits dividing the cone-shaped segment into a plurality of flaps.

23. The apparatus according to claim 22, wherein the plurality of flaps are biased apart from one another, and the guidewire passageway further comprises an aperture that is adapted to receive the cone-shaped segment and bring the flaps in contact with one another and thereby constrict the aperture around the guidewire.

24. The apparatus according to claim 14, wherein the guide member further comprises:
an aperture providing transverse access to the guidewire passageway from outside the guide member;
a mechanical switch extending through the aperture and comprising a first end adapted to press against the clamping body with a force that is sufficient to clamp the guidewire.

25. The apparatus according to claim 24, wherein the mechanical switch is biased away from the clamping body and requires an unbiasing force to press against the clamping body.

26. The apparatus according to claim 25, wherein the mechanical switch is biased using at least one spring.
